(19) United States
(12) Patent Application Publication
(10) Pub. No.: US 2019/0087603 A1
(43) Pub. Date: Mar. 21, 2019

(54) MOBILE DEVICE AND METHOD FOR ENABLING PATIENTS TO CONTROL ACCESS TO THEIR MEDICAL RECORDS

(71) Applicants: Omer Dror, Tel Aviv (IL); Ron Zass, Kiryat Tivon (IL)

(72) Inventors: Omer Dror, Tel Aviv (IL); Ron Zass, Kiryat Tivon (IL)

(21) Appl. No.: 16/194,341

(22) Filed: Nov. 18, 2018

Related U.S. Application Data
(60) Provisional application No. 62/588,321, filed on Nov. 18, 2017.

(51) Int. Cl. G06F 21/62; G16H 10/60; G06K 9/32; H04W 4/80

(52) U.S. Cl. CPC G06F 21/6245; H04W 4/80; G06K 9/3241; G16H 10/60

(57) ABSTRACT

System and method for enabling patients to control access to their medical records are provided. Information identifying one or more medical care providers may be received. Further, a request may be provided to a user to grant permission to the one or more medical care providers to access at least part of a plurality of medical records of the patient. A response to the request may be received from the user. Based on the response, the permission to access the at least part of the plurality of medical records of the patient may be granted to at least part of the one or more medical care providers.

### CROSS REFERENCES TO RELATED APPLICATIONS

This application claims the benefit of priority of U.S. Provisional Patent Application No. 62/588,321, filed on Nov. 18, 2017, the disclosures of which incorporated herein by reference in their entirety.

### BACKGROUND

### Technological Field

The disclosed embodiments generally relate to systems and methods for medical records management. More particularly, the disclosed embodiments relate to systems and methods for medical records management that allow sharing of medical information.

### Background Information

Numerous medical records are created, read and edited by vast number of medical care providers. Sharing medical information among medical care providers may prove challenging.

### SUMMARY

In some embodiments, systems and methods for medical records management are provided.

In some embodiments, information identifying one or more medical care providers may be received. Further, a request may be provided to a user to grant permission to the one or more medical care providers to access at least part of a plurality of medical records of the patient. A response to the request may be received from the user. Based on the response, the permission to access the at least part of the plurality of medical records of the patient may be granted to at least part of the one or more medical care providers.

### BRIEF DESCRIPTION OF THE DRAWINGS

FIGS. 1A and 1B are illustrations of possible systems for providing permissions to access medical records.

FIGS. 2A and 2B are block diagrams illustrating some possible implementations of an apparatus.

FIG. 3 is a block diagram illustrating a possible implementation of a server.

FIGS. 4A and 4B are block diagrams illustrating some possible implementations of a cloud platform.

FIG. 5 is a block diagram illustrating a possible implementation of a computational node.

FIG. 6 illustrates an exemplary embodiment of a memory storing a plurality of modules.

FIG. 7 illustrates an example of a process for providing permissions to access medical records.

FIG. 8 illustrates an example of a visual user interface.

### DESCRIPTION

Unless specifically stated otherwise, as apparent from the following discussions, it is appreciated that throughout the specification discussions utilizing terms such as “processing”, “calculating”, “computing”, “determining”, “generating”, “setting”, “configuring”, “selecting”, “defining”, “applying”, “obtaining”, “monitoring”, “providing”, “identifying”, “segmenting”, “classifying”, “analyzing”, “associating”, “extracting”, “storing”, “receiving”, “transmitting”, or the like, include action and/or processes of a computer that manipulate and/or transform data into other data, said data represented as physical quantities, for example such as electronic quantities, and/or said data representing the physical objects. The terms “computer”, “processor”, “controller”, “processing unit”, “computing unit”, and “processing module” should be expansively construed to cover any kind of electronic device, component or unit with data processing capabilities, including, by way of non-limiting example, a personal computer, a wearable computer, a tablet, a smartphone, a server, a computing system, a cloud computing platform, a communication device, a processor (for example, digital signal processor (DSP), an image signal processor (ISR), a microcontroller, a field programmable gate array (FPGA), an application specific integrated circuit (ASIC), a central processing unit (CPA), a graphics processing unit (GPU), a visual processing unit (VPU), and so on), possibly with embedded memory, a single core processor, a multi core processor, a core within a processor, any other electronic computing device, or any combination of the above.

The operations in accordance with the teachings herein may be performed by a computer specially constructed or programmed to perform the described functions.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) may be included in at least one embodiment of the presently disclosed subject matter. Thus the appearance of the phrase “one case”, “some cases”, “other cases” or variants thereof does not necessarily refer to the same embodiment(s). As used herein, the term “and/or” includes any and all combinations of one or more of the associated listed items.

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

The term “image sensor” is recognized by those skilled in the art and refers to any device configured to capture images, a sequence of images, videos, and so forth. This includes sensors that convert optical input into images, where optical input can be visible light (like in a camera), radio waves, microwaves, terahertz waves, ultraviolet light, infrared light, x-rays, gamma rays, and/or any other light spectrum. This also includes both 2D and 3D sensors. Examples of image sensor technologies may include: CCD, CMOS, NMOS, and so forth. 3D sensors may be implemented using different technologies, including: stereo camera, active stereo camera, time of flight camera, structured light camera, radar, range image camera, and so forth.

In embodiments of the presently disclosed subject matter, one or more stages illustrated in the figures may be executed in a different order and/or one or more groups of stages may be executed simultaneously and vice versa. The figures illustrate a general schematic of the system architecture in accordance embodiments of the presently disclosed subject matter. Each module in the figures can be made up of any combination of software, hardware and/or firmware that performs the functions as defined and explained herein. The modules in the figures may be centralized in one location or dispersed over more than one location.

It should be noted that some examples of the presently disclosed subject matter are not limited in application to the details of construction and the arrangement of the components set forth in the following description or illustrated in the drawings. The invention can be capable of other embodiments or of being practiced or carried out in various ways. Also, it is to be understood that the phraseology and terminology employed herein is for the purpose of description and should not be regarded as limiting.

In this document, an element of a drawing that is not described within the scope of the drawing and is labeled with a numeral that has been described in a previous drawing may have the same use and description as in the previous drawings.

The drawings in this document may not be to any scale. Different figures may use different scales and different scales can be used even within the same drawing, for example different scales for different views of the same object or different scales for the two adjacent objects.

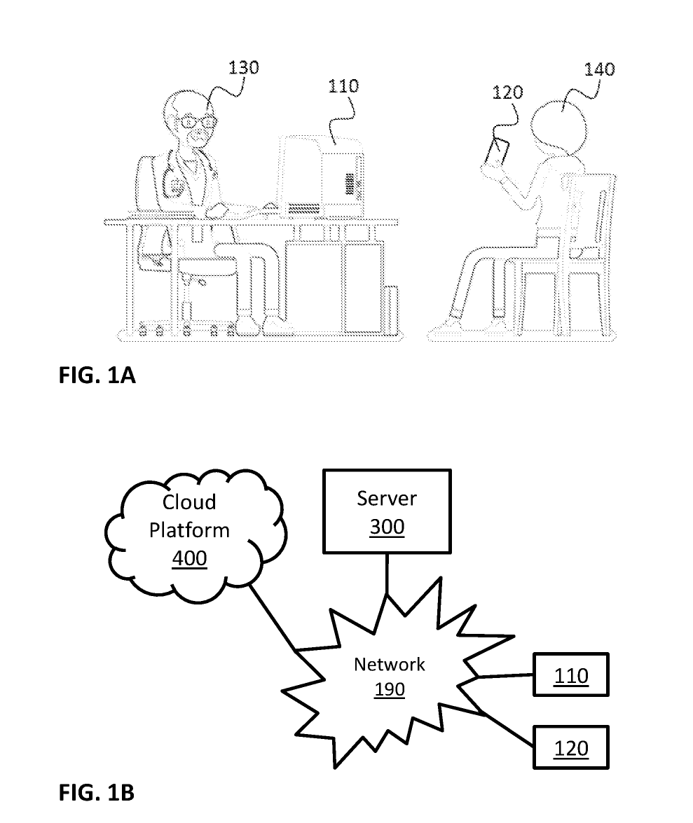

FIG. 1A is an illustration of a possible system for providing permissions to access medical records. In this example, patient 140 may meet medical care provider 130, for example by visiting medical care provider 130, by meeting outside the office of medical care provider 130, and so forth. Medical care provider 130 may use electronic device 110, and patient 140 may use electronic device 120. Some examples of possible implementations of electronic devices 110 and/or 120 may include computerized devices, wearable computers, smart glasses, mobile devices, mobile phones, smartphones, smart watches, tablets, personal computers (PC), and so forth. While FIG. 1A depicts medical care provider 130 in the same room with patient 140, in other examples the medical care provider 130 and the patient 140 may be in different rooms, possibly located a great distance from one another. The medical care provider 130 and patient 140 may communicate with one another through face to face interaction, by using mediating technology (such as phone, video call, messaging software, emails, fax, and so forth), or not communicate with one another at all. In some examples, patient 140 may be accompanied by another person or be substituted by a person representing the patient, such as a legal guardian of the patient, a care giver of the patient, a family member of the patient, and so forth. In such cases, electronic device 120 may be used by the person accompanying or substituting the patient.

FIG. 1B is an illustration of a possible system for providing permissions to access medical records. In this example, electronic device 110 associated with a medical care provider (such as medical care provider 130) and/or electronic device 120 associated with a patient (such as patient 140) may be connected to a communication network 190. In some examples, additional devices may be connected to communication network 190, such as server 300, cloud platform 400, remote storage, network attached storage (NAS), and so forth.

Some examples of communication network 190 may include the Internet, phone networks, cellular networks, satellite communication networks, private communication networks, virtual private networks (VPN), wireless network, wired network, point-to-point communication between two devices, and so forth. Electronic device 110 may communicate with electronic device 120 through communication network 190 and/or directly. Electronic device 110 and/or electronic device 120 may communicate with server 300 and/or cloud platform 400 and/or remote storage and/or network attached storage (NAS) through communication network 190 and/or directly.

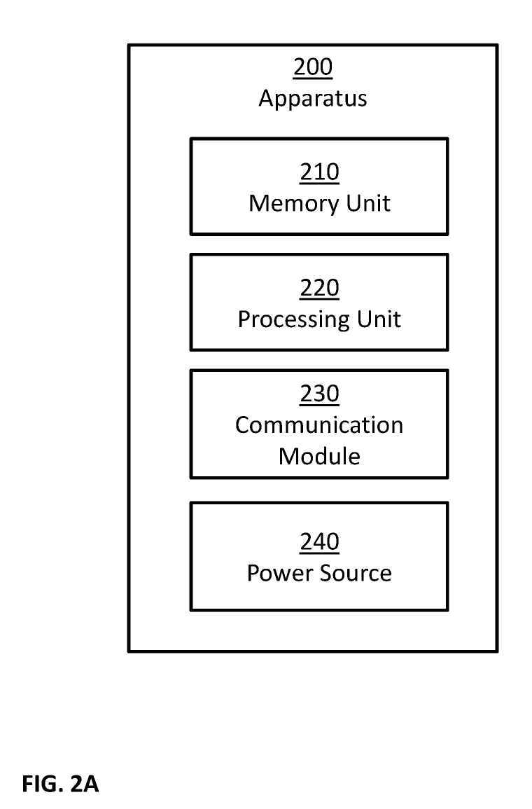

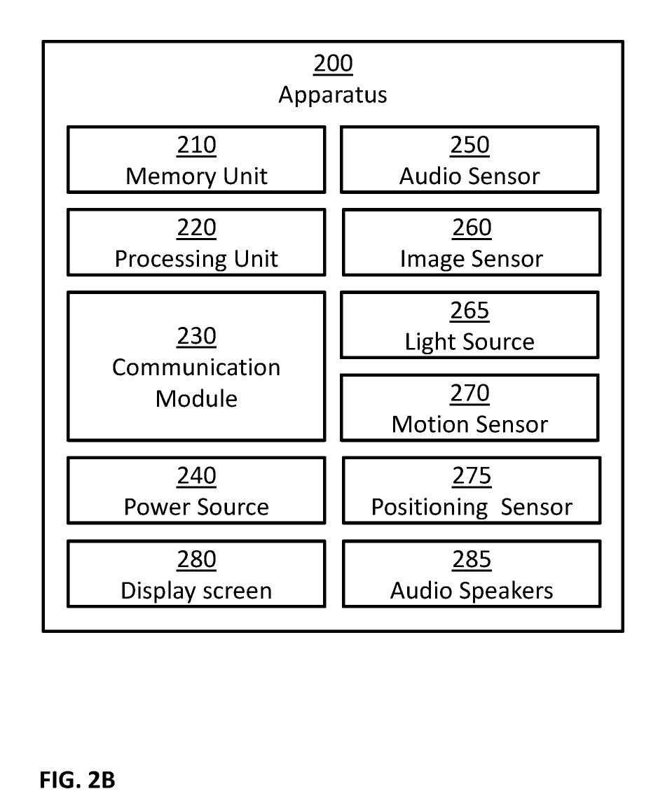

Some possible implementations of electronic device 110 and/or electronic device 120 may include apparatus 200 as described in FIGS. 2A and 2B. In some embodiments, electronic device 110 and/or electronic device 120 and/or apparatus 200 may be mobile devices.

FIG. 2A is a block diagram illustrating a possible implementation of apparatus 200. In this example, apparatus 200 may comprise: one or more memory units 210, one or more processing units 220, one or more communication modules 230, and one or more power sources 240. In some implementations, apparatus 200 may comprise additional components, while some components listed above may be excluded.

FIG. 2B is a block diagram illustrating a possible implementation of apparatus 200. In this example, apparatus 200 may comprise: one or more memory units 210, one or more processing units 220, one or more communication modules 230, one or more power sources 240, one or more audio sensors 250, one or more image sensors 260, one or more light sources 265, one or more motion sensors 270, one or more positioning sensors 275, one or more display screens 280, and one or more audio speakers 285. In some implementations, apparatus 200 may comprise additional components, while some components listed above may be excluded. For example, in some implementations apparatus 200 may also comprise at least one of the following: one or more barometers; one or more user input devices; one or more output devices; and so forth. In another example, in some implementations at least one of the following may be excluded from apparatus 200: memory units 210, communication modules 230, power sources 240, audio sensors 250, image sensors 260, light sources 265, motion sensors 270, positioning sensors 275, display screens 280 and audio speakers 285.

In some embodiments, one or more power sources 240 may be configured to power apparatus 200 and/or power server 300 and/or power cloud platform 400 and/or power computational node 500. Possible implementation examples of power sources 240 may include: one or more electric batteries; one or more capacitors; one or more connections to external power sources; one or more power convertors; any combination of the above; and so forth.

In some embodiments, the one or more processing units 220 may be configured to execute software programs. For example, processing units 220 may be configured to execute software programs stored on the memory units 210. In some cases, the executed software programs may store information in memory units 210. In some cases, the executed software programs may retrieve information from the memory units 210. Possible implementation examples of the processing units 220 may include: one or more single core processors, one or more multicore processors; one or more controllers; one or more application processors; one or more system on a chip processors; one or more central processing units; one or more graphical processing units; one or more neural processing units; any combination of the above; and so forth.

In some embodiments, the one or more communication modules 230 may be configured to receive and transmit information. For example, control signals may be transmitted and/or received through communication modules 230. In another example, information received though communication modules 230 may be stored in memory units 210. In an additional example, information retrieved from memory units 210 may be transmitted using communication modules 230. In another example, input data may be transmitted and/or received using communication modules 230. Examples of such input data may include: input data inputted by a user using user input devices; information captured using one or more sensors; and so forth. Examples of such sensors may include: audio sensors 250; image sensors 260; motion sensors 270; positioning sensors 275; chemical sensors; temperature sensors; barometers; and so forth.

In some embodiments, the one or more audio sensors 250 may be configured to capture audio by converting sounds to digital information. Some examples of audio sensors 250 may include: microphones, unidirectional microphones, bidirectional microphones, cardioid microphones, omnidirectional microphones, onboard microphones, wired microphones, wireless microphones, any combination of the above, and so forth. In some examples, the captured audio may be stored in memory units 210. In some additional examples, the captured audio may be transmitted using communication modules 230, for example to other computerized devices, such as server 300, cloud platform 400, computational node 500, and so forth. In some examples, processing units 220 may control the above processes. For example, processing units 220 may control at least one of: capturing of the audio; storing the captured audio; transmitting of the captured audio; and so forth. In some cases, the captured audio may be processed by processing units 220. For example, the captured audio may be compressed by processing units 220; possibly followed: by storing the compressed captured audio in memory units 210; by transmitted the compressed captured audio using communication modules 230; and so forth. In another example, the captured audio may be processed using speech recognition algorithms. In another example, the captured audio may be processed using speaker recognition algorithms.

In some embodiments, the one or more image sensors 260 may be configured to capture visual information by converting light to: images; sequence of images; videos; 3D images; sequence of 3D images; 3D videos; and so forth. In some examples, the captured visual information may be stored in memory units 210. In some additional examples, the captured visual information may be transmitted using communication modules 230, for example to other computerized devices, such as server 300, cloud platform 400, computational node 500, and so forth. In some examples, processing units 220 may control the above processes. For example, processing units 220 may control at least one of: capturing of the visual information; storing the captured visual information; transmitting of the captured visual information; and so forth. In some cases, the captured visual information may be processed by processing units 220. For example, the captured visual information may be compressed by processing units 220; possibly followed: by storing the compressed captured visual information in memory units 210; by transmitted the compressed captured visual information using communication modules 230; and so forth. In another example, the captured visual information may be processed in order to: detect objects, detect events, detect action, detect face, detect people, recognize person, and so forth.

In some embodiments, the one or more light sources 265 may be configured to emit light, for example in order to enable better image capturing by image sensors 260. In some examples, the emission of light may be coordinated with the capturing operation of image sensors 260. In some examples, the emission of light may be continuous. In some examples, the emission of light may be performed at selected times. The emitted light may be visible light, infrared light, x-rays, gamma rays, and/or in any other light spectrum. In some examples, image sensors 260 may capture light emitted by light sources 265, for example in order to capture 3D images and/or 3D videos using active stereo method.

In some embodiments, the one or more motion sensors 270 may be configured to perform at least one of the following: detect motion of objects in the environment of apparatus 200; measure the velocity of objects in the environment of apparatus 200; measure the acceleration of objects in the environment of apparatus 200; detect motion of apparatus 200; measure the velocity of apparatus 200; measure the acceleration of apparatus 200; and so forth. In some implementations, the one or more motion sensors 270 may comprise one or more accelerometers configured to detect changes in proper acceleration and/or to measure proper acceleration of apparatus 200. In some implementations, the one or more motion sensors 270 may comprise one or more gyroscopes configured to detect changes in the orientation of apparatus 200 and/or to measure information related to the orientation of apparatus 200. In some implementations, motion sensors 270 may be implemented using image sensors 260, for example by analyzing images captured by image sensors 260 to perform at least one of the following tasks: track objects in the environment of apparatus 200; detect moving objects in the environment of apparatus 200; measure the velocity of objects in the environment of apparatus 200; measure the acceleration of objects in the environment of apparatus 200; measure the velocity of apparatus 200, for example by calculating the egomotion of image sensors 260; measure the acceleration of apparatus 200, for example by calculating the egomotion of image sensors 260; and so forth. In some implementations, motion sensors 270 may be implemented using image sensors 260 and light sources 265, for example by implementing a LIDAR using image sensors 260 and light sources 265. In some implementations, motion sensors 270 may be implemented using one or more RADARs. In some examples, information captured using motion sensors 270: may be stored in memory units 210, may be processed by processing units 220, may be transmitted and/or received using communication modules 230, and so forth.

In some embodiments, the one or more positioning sensors 275 may be configured to obtain positioning information of apparatus 200, to detect changes in the position of apparatus 200, and/or to measure the position of apparatus 200. In some examples, positioning sensors 275 may be implemented using one of the following technologies: Global Positioning System (GPS), GLObal NAvigation Satellite System (GLONASS), Galileo global navigation system, BeiDou navigation system, other Global Navigation Satellite Systems (GNSS), Indian Regional Navigation Satellite System (IRNSS), Local Positioning Systems (LPS), Real-Time Location Systems (RTLS), Indoor Positioning System (IPS), Wi-Fi based positioning systems, cellular triangulation, and so forth. In some examples, information captured using positioning sensors 275 may be stored in memory units 210, may be processed by processing units 220, may be transmitted and/or received using communication modules 230, and so forth.

In some embodiments, the one or more chemical sensors may be configured to perform at least one of the following: measure chemical properties in the environment of apparatus 200; measure changes in the chemical properties in the environment of apparatus 200; detect the present of chemicals in the environment of apparatus 200; measure the concentration of chemicals in the environment of apparatus 200. Examples of such chemical properties may include: pH level, toxicity, temperature, and so forth. Examples of such chemicals may include: electrolytes, particular enzymes, particular hormones, particular proteins, smoke, carbon dioxide, carbon monoxide, oxygen, ozone, hydrogen, hydrogen sulfide, and so forth. In some examples, information captured using chemical sensors may be stored in memory units 210, may be processed by processing units 220, may be transmitted and/or received using communication modules 230, and so forth.

In some embodiments, the one or more temperature sensors may be configured to detect changes in the temperature of the environment of apparatus 200 and/or to measure the temperature of the environment of apparatus 200. In some examples, information captured using temperature sensors may be stored in memory units 210, may be processed by processing units 220, may be transmitted and/or received using communication modules 230, and so forth.

In some embodiments, the one or more barometers may be configured to detect changes in the atmospheric pressure in the environment of apparatus 200 and/or to measure the atmospheric pressure in the environment of apparatus 200. In some examples, information captured using the barometers may be stored in memory units 210, may be processed by processing units 220, may be transmitted and/or received using communication modules 230, and so forth.

In some embodiments, the one or more user input devices may be configured to allow one or more users to input information. In some examples, user input devices may comprise at least one of the following: a keyboard, a mouse, a touch pad, a touch screen, a joystick, a microphone, an image sensor, and so forth. In some examples, the user input may be in the form of at least one of: text, sounds, speech, hand gestures, body gestures, tactile information, and so forth. In some examples, the user input may be stored in memory units 210, may be processed by processing units 220, may be transmitted and/or received using communication modules 230, and so forth.

In some embodiments, the one or more user output devices may be configured to provide output information to one or more users. In some examples, such output information may comprise of at least one of: notifications, feedbacks, reports, and so forth. In some examples, user output devices may comprise at least one of: one or more audio output devices (such as audio speakers 285); one or more textual output devices; one or more visual output devices (such as display screens 280); one or more tactile output devices; and so forth. In some examples, the one or more audio output devices may be configured to output audio to a user, for example through: a headset, audio speakers 285, and so forth. In some examples, the one or more visual output devices may be configured to output visual information to a user, for example through: a display screen (such as display screens 280), an augmented reality display system, a printer, a LED indicator, and so forth. In some examples, display screen 280 may include a touch screen configured to display information to a user and receive touch input from the user. In some examples, the one or more tactile output devices may be configured to output tactile feedbacks to a user, for example through vibrations, through motions, by applying forces, and so forth. In some examples, the output may be provided: in real time, offline, automatically, upon request, and so forth. In some examples, the output information may be read from memory units 210, may be provided by a software executed by processing units 220, may be transmitted and/or received using communication modules 230, and so forth.

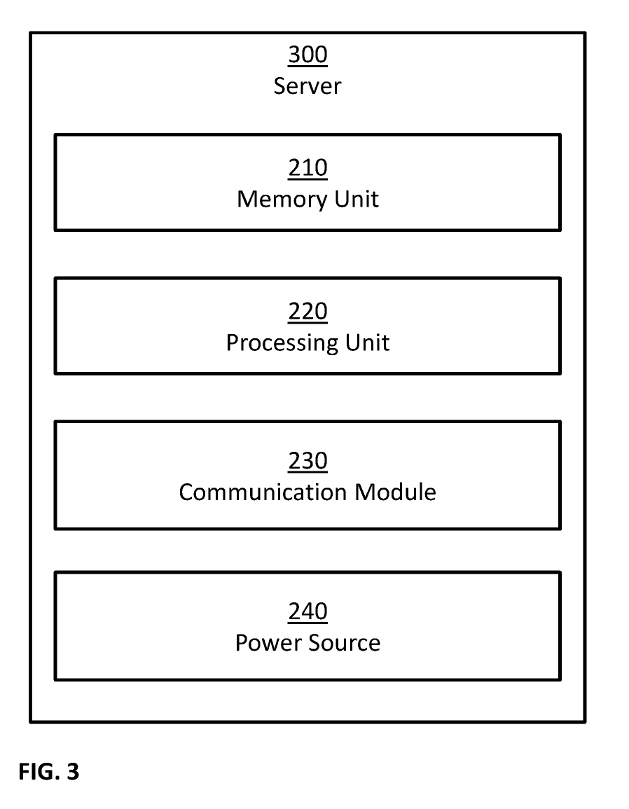

FIG. 3 is a block diagram illustrating a possible implementation of server 300. In this example, server 300 may comprise: one or more memory units 210, one or more processing units 220, one or more communication modules 230, and one or more power sources 240. In some implementations, server 300 may comprise additional components, while some components listed above may be excluded. For example, in some implementations server 300 may also comprise at least one of the following: one or more user input devices; one or more output devices; and so forth. In another example, in some implementations at least one of the following may be excluded from server 300: memory units 210, communication modules 230, and power sources 240.

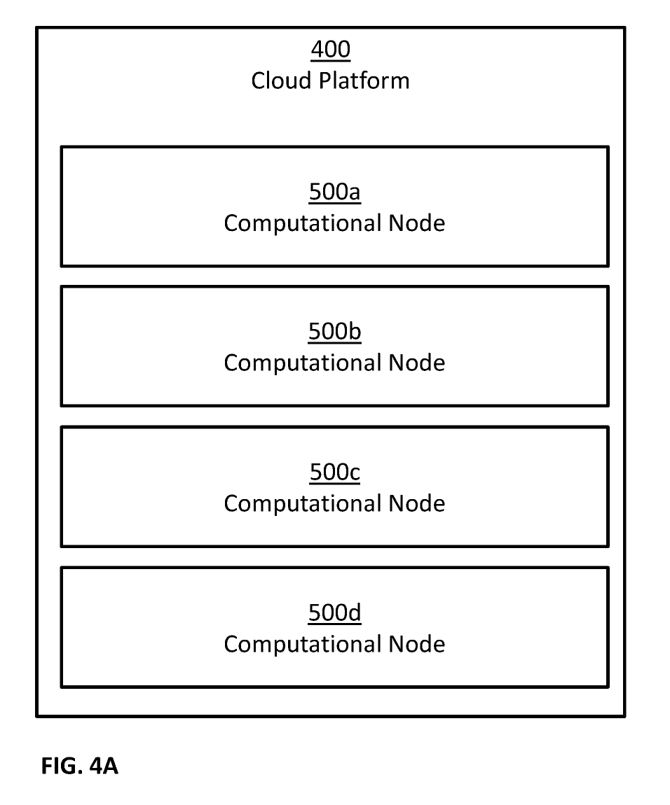

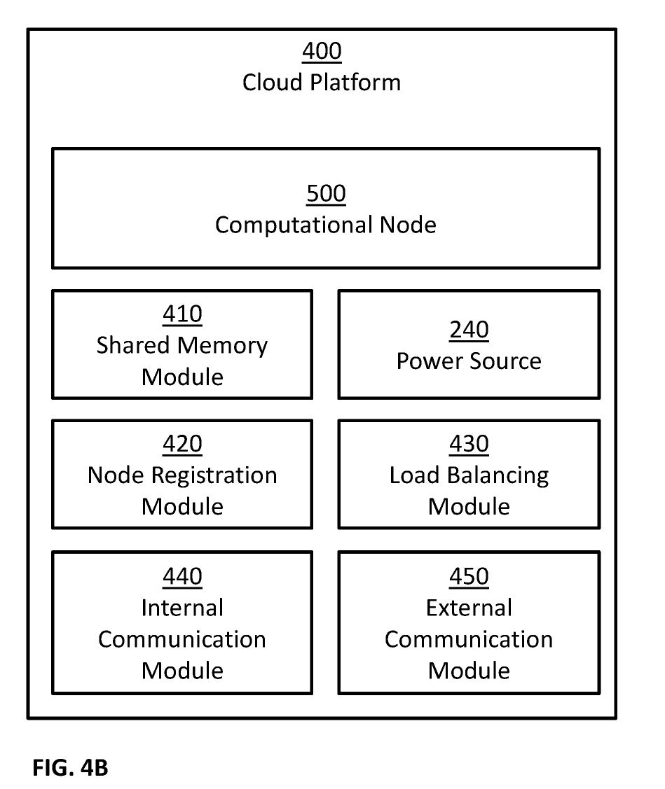

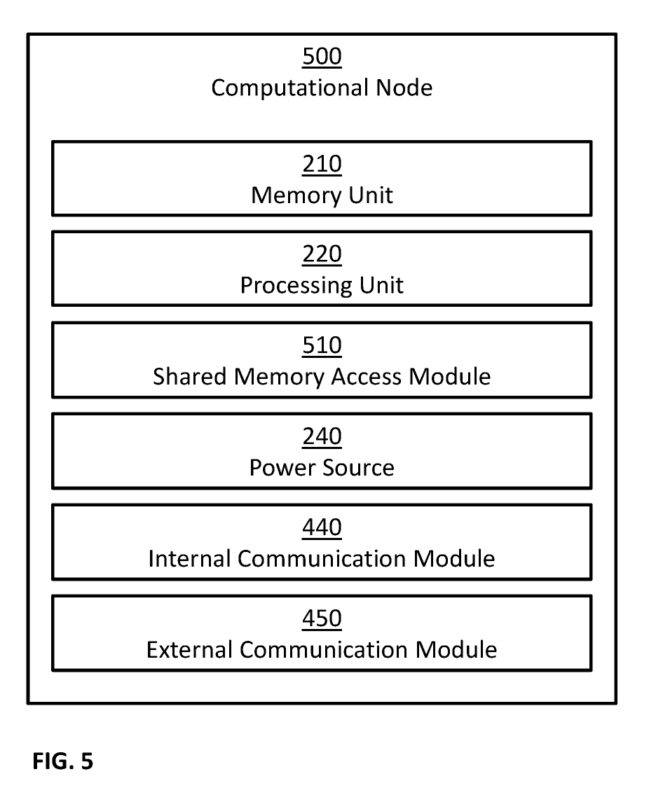

FIG. 4A is a block diagram illustrating a possible implementation of cloud platform 400. In this example, cloud platform 400 may comprise computational node 500a, computational node 500b, computational node 500c and computational node 500d. In some examples, a possible implementation of computational nodes 500a, 500b, 500c and 500d may comprise server 300 as described in FIG. 3. In some examples, a possible implementation of computational nodes 500a, 500b, 500c and 500d may comprise computational node 500 as described in FIG. 5.

FIG. 4B is a block diagram illustrating a possible implementation of cloud platform 400. In this example, cloud platform 400 may comprise: one or more computational nodes 500, one or more shared memory modules 410, one or more power sources 240, one or more node registration modules 420, one or more load balancing modules 430, one or more internal communication modules 440, and one or more external communication modules 450. In some implementations, cloud platform 400 may comprise additional components, while some components listed above may be excluded. For example, in some implementations cloud platform 400 may also comprise at least one of the following: one or more user input devices; one or more output devices; and so forth. In another example, in some implementations at least one of the following may be excluded from cloud platform 400: shared memory modules 410, power sources 240, node registration modules 420, load balancing modules 430, internal communication modules 440, and external communication modules 450.

FIG. 5 is a block diagram illustrating a possible implementation of computational node 500. In this example, computational node 500 may comprise: one or more memory units 210, one or more processing units 220, one or more shared memory access modules 510, one or more power sources 240, one or more internal communication modules 440, and one or more external communication modules 450. In some implementations, computational node 500 may comprise additional components, while some components listed above may be excluded. For example, in some implementations computational node 500 may also comprise at least one of the following: one or more user input devices; one or more output devices; and so forth. In another example, in some implementations at least one of the following may be excluded from computational node 500: memory units 210, shared memory access modules 510, power sources 240, internal communication modules 440, and external communication modules 450.

In some embodiments, internal communication modules 440 and external communication modules 450 may be implemented as a combined communication module, such as communication modules 230. In some embodiments, one possible implementation of cloud platform 400 may comprise server 300. In some embodiments, one possible implementation of computational node 500 may comprise server 300. In some embodiments, one possible implementation of shared memory access modules 510 may comprise using internal communication modules 440 to send information to shared memory modules 410 and/or receive information from shared memory modules 410. In some embodiments, node registration modules 420 and load balancing modules 430 may be implemented as a combined module.

In some embodiments, the one or more shared memory modules 410 may be accessed by more than one computational node. Therefore, shared memory modules 410 may allow information sharing among two or more computational nodes 500. In some embodiments, the one or more shared memory access modules 510 may be configured to enable access of computational nodes 500 and/or the one or more processing units 220 of computational nodes 500 to shared memory modules 410. In some examples, computational nodes 500 and/or the one or more processing units 220 of computational nodes 500, may access shared memory modules 410, for example using shared memory access modules 510, in order to perform at least one of: executing software programs stored on shared memory modules 410, store information in shared memory modules 410, retrieve information from the shared memory modules 410.

In some embodiments, the one or more node registration modules 420 may be configured to track the availability of the computational nodes 500. In some examples, node registration modules 420 may be implemented as: a software program, such as a software program executed by one or more of the computational nodes 500; a hardware solution; a combined software and hardware solution; and so forth. In some implementations, node registration modules 420 may communicate with computational nodes 500, for example using internal communication modules 440. In some examples, computational nodes 500 may notify node registration modules 420 of their status, for example by sending messages: at computational node 500 startup; at computational node 500 shutdown; at constant intervals; at selected times; in response to queries received from node registration modules 420; and so forth. In some examples, node registration modules 420 may query about computational nodes 500 status, for example by sending messages: at node registration module 420 startup; at constant intervals; at selected times; and so forth.

In some embodiments, the one or more load balancing modules 430 may be configured to divide the work load among computational nodes 500. In some examples, load balancing modules 430 may be implemented as: a software program, such as a software program executed by one or more of the computational nodes 500; a hardware solution; a combined software and hardware solution; and so forth. In some implementations, load balancing modules 430 may interact with node registration modules 420 in order to obtain information regarding the availability of the computational nodes 500. In some implementations, load balancing modules 430 may communicate with computational nodes 500, for example using internal communication modules 440. In some examples, computational nodes 500 may notify load balancing modules 430 of their status, for example by sending messages: at computational node 500 startup; at computational node 500 shutdown; at constant intervals; at selected times; in response to queries received from load balancing modules 430; and so forth. In some examples, load balancing modules 430 may query about computational nodes 500 status, for example by sending messages: at load balancing module 430 startup; at constant intervals; at selected times; and so forth.

In some embodiments, the one or more internal communication modules 440 may be configured to receive information from one or more components of cloud platform 400, and/or to transmit information to one or more components of cloud platform 400. For example, control signals and/or synchronization signals may be sent and/or received through internal communication modules 440. In another example, input information for computer programs, output information of computer programs, and/or intermediate information of computer programs, may be sent and/or received through internal communication modules 440. In another example, information received though internal communication modules 440 may be stored in memory units 210, in shared memory units 410, and so forth. In an additional example, information retrieved from memory units 210 and/or shared memory units 410 may be transmitted using internal communication modules 440. In another example, input data may be transmitted and/or received using internal communication modules 440. Examples of such input data may include input data inputted by a user using user input devices.

In some embodiments, the one or more external communication modules 450 may be configured to receive and/or to transmit information. For example, control signals may be sent and/or received through external communication modules 450. In another example, information received though external communication modules 450 may be stored in memory units 210, in shared memory units 410, and so forth. In an additional example, information retrieved from memory units 210 and/or shared memory units 410 may be transmitted using external communication modules 450. In another example, input data may be transmitted and/or received using external communication modules 450. Examples of such input data may include: input data inputted by a user using user input devices; information captured from the environment of apparatus 200 using one or more sensors; and so forth. Examples of such sensors may include: audio sensors 250; image sensors 260; motion sensors 270; positioning sensors 275; chemical sensors; temperature sensors; barometers; and so forth.

In some embodiments, some entities (such as patients, relatives of patients, medical care providers, insurers, etc.) may use computerized devices (such as electronic device 110, electronic device 120, apparatus 200, server 300, computational node 500, cloud platform 400, etc.) to perform part and/or all of their functions and/or duties. For example, the entities may use computerized devices to store and/or access and/or process data (some examples of such data may include medical records 610, scheduling records 620, financial records 630, insurance records 640, and/or permissions 650 described below), to communicate (for example over communication network 190), and so forth.

In some examples, patients, such as patient 140, may include one or more individuals that received and/or receiving and/or are about to receive medical care.

In some examples, relatives may include one or more individuals that have some bearing on the medical care of at least one patient. For example, relatives may comprise one or more of a family member of a patient, a friend of a patient, a legal guardian of a patient, a next of kin of a patient, a non-medical care giver of a patient, and so forth.

In some examples, medical care providers, such as medical care provider 130, may include one or more individual and/or one or more institutes that provides (in the past and/or present and/or future) medical care to patients. For example, medical care providers may include one or more medical care professionals (such as medical doctors, nurses, therapists, stuff of medical care institutes, etc.), one or more medical care institutes (such as hospitals, clinics, labs, etc.), and so forth.

In some embodiments, insurers may include one or more individuals and/or one or more institutes that cover medical expenses (such as medical expenses of patients and/or of medical care providers) and/or insures medical care providers for malpractice costs. In some examples, insurers may include insurance firms and/or government agencies.

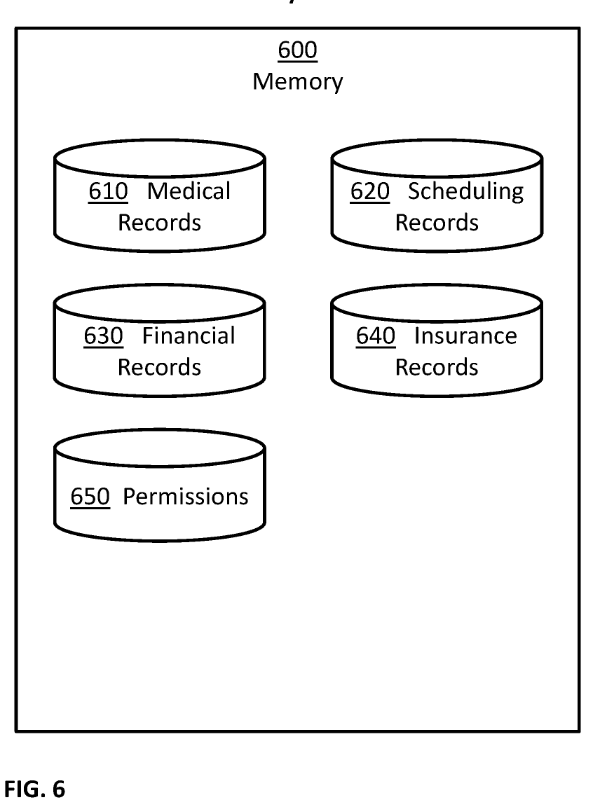

FIG. 6 illustrates an exemplary embodiment of memory 600 storing a plurality of modules. In some examples, memory 600 may be separate from and/or integrated with memory units 210, separate from and/or integrated with memory units 410, and so forth. In some examples, memory 600 may be included in a single device, for example in electronic device 110, in electronic device 120, in apparatus 200, in server 300, in cloud platform 400, in computational node 500, and so forth. In some examples, memory 600 may be distributed across several devices. Memory 600 may store more or fewer modules than those shown in FIG. 6. In this example, memory 600 may comprise: medical records 610, scheduling records 620, financial records 630, insurance records 640, and permissions 650.

In some embodiments, at least part of medical records 610 scheduling records 620, financial records 630, insurance records 640, and/or permissions 650 may be stored in a public database, in a public ledger, in a blockchain, in a computerized devices (such as electronic device 110, electronic device 120, apparatus 200, server 300, cloud platform 400, computational node 500, etc.), in a storage devices (such as remote storage, network attached storage, etc.), and so forth. In some examples, medical records 610, scheduling records 620, financial records 630, insurance records 640, and/or permissions 650 may be stored in single database and/or a single blockchain and/or a single site and/or a single device, while in other examples medical records 610 scheduling records 620, financial records 630, insurance records 640, and/or permissions 650 may be distributed among a number of databases and/or blockchains and/or sites and/or devices. In some examples, medical records associated with a single entity (such as patient, relative, medical care provider, insurer, etc.) may be stored in single database and/or in a single blockchain and/or in a single site and/or in a single device, while in other examples the medical records associated with the entity may be distributed among a number of databases and/or blockchains and/or sites and/or devices.

In some embodiments, medical records 610 may comprise medical records of one or more patients, medical records created and/or used by one or more medical care providers, medical records associated with one or more patients and/or with one or more medical care providers insured by one or more insurers, and so forth. In some examples, medical records 610 may comprise medical information, such as information regarding medical conditions, medical care, medical treatment, electronic health records, genome data, and so forth.

In some embodiments, scheduling records 620 may comprise scheduling related information associated with patients and/or relatives and/or medical care providers and/or insurers. The scheduling related information may relate to past and/or present and/or future events. For example, scheduling records 620 may comprise time and date information for an appointment, for a lab test, for a medical exam, for a medical checkup, for a reminder related to medical care, and so forth.

In some embodiments, financial records 630 may comprise financial information associated with patients and/or relatives and/or medical care providers and/or insurers. The financial information may relate to past, present, and/or future budget, costs, bills, coverage obligations, and/or payments associated with medical care.

In some embodiments, insurance records 640 may comprise insurance information associated with patients and/or relatives and/or medical care providers and/or insurers. The insurance information may include past, present and/or future coverage information, insurance claims, insurance payments, and so forth, associated with medical care.

In some embodiments, permissions 650 may specify which entities (such as patients, relatives, medical care providers, insurers, etc.) may create and/or edit and/or access which records (such as medical records 610, scheduling records 620, financial records 630, insurance records 640, permissions 650, and so forth). For example, permissions 650 may comprise a group or entities allowed to create and/or edit and/or access selected records, a group or entities prohibited from creating and/or editing and/or accessing selected records, and so forth. For example, the selected records above may be specified as a group of records, as a rule defining a group of records, as the records associated with selected entities, and so forth. In some examples, permissions 650 may further specify which entities are allowed to grant which permission to which other entities regarding which records.

In some embodiments, medical care providers may create and/or edit records of a patient (such as medical records 610 scheduling records 620, financial records 630, insurance records 640, permissions 650, etc.). These records may be accessed and/or edited by the patient, by some relatives of the patient, by other medical care providers treating the patient, by insurers of the patient and/or the medical care provider, and so forth.

In some embodiments, an insurer may access and/or edit and/or create records (such as medical records 610 scheduling records 620, financial records 630, insurance records 640, permissions 650, etc.) of entities (such as patients, relatives, medical care providers, other insurers, etc.) insured by the insurer.

In some embodiments, access to a record and/or creation of a record and/or edition to a record may be recorded, for example in a log, in the accessed and/or created and/or edited record, and so forth.

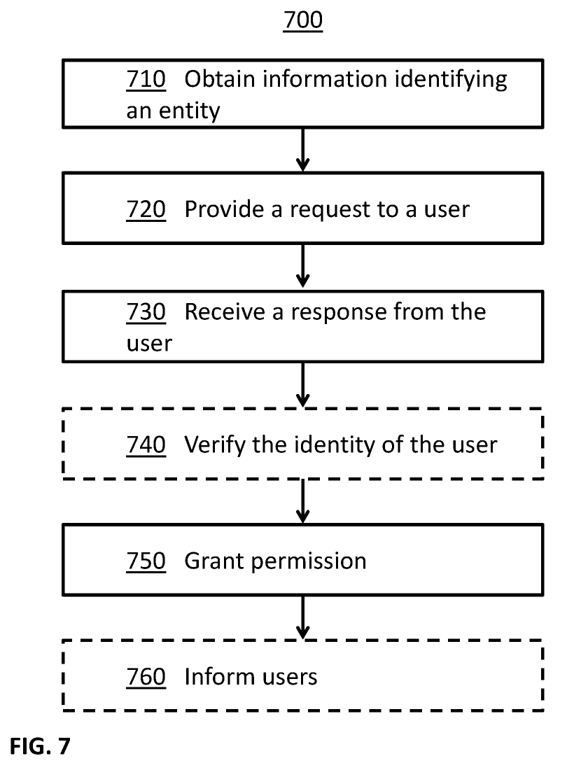

FIG. 7 illustrates an example of a process 700 for providing permissions to access medical records. In some examples, process 700, as well as all individual steps therein, may be performed by various aspects of: electronic device 110, electronic device 120, apparatus 200, server 300, cloud platform 400, computational node 500, mobile device, and so forth. For example, process 700 may be performed by processing units 220, executing software instructions stored within memory units 210 and/or within shared memory modules 410. In another example, process 700 may be performed by and/or using a mobile device associated with a patient. In this example, process 700 may comprise: obtaining information identifying an entity (Step 710); providing a request to a user (Step 720); receiving response from the user (Step 730); verifying the user identity (Step 740); granting permission (Step 750); and informing users (Step 760). In some implementations, process 700 may comprise one or more additional steps, while some of the steps listed above may be modified or excluded. For example, Step 740 and/or Step 760 may be excluded from process 700. In some implementations, one or more steps illustrated in FIG. 7 may be executed in a different order and/or one or more groups of steps may be executed simultaneously and vice versa. For example, Step 740 may be executed before, after and/or simultaneously with Steps 710, 720 and/or 730. In another example, Step 760 may be executed after and/or simultaneously with Step 750. Examples of possible execution manners of process 700 may include: continuous execution, returning to the beginning of the process once the process normal execution ends; periodically execution, executing the process at selected times; execution upon the detection of a trigger, where examples of such trigger may include trigger from a user, trigger from another process, etc.; any combination of the above; and so forth.

In some embodiments, obtaining information identifying an entity (Step 710) may comprise obtaining information identifying a patient (such as patient 140), a relative of a patient, a medical care provider, a medical care institute, an insurer, and so forth. Furthermore, additional information may be obtained, such as information about the entity, information about the location of the entity, information indicating that a medical care provider and a patient are present in the same space (such as a clinic, an office, a room, etc.), information indicating that a medical care provider and a patient are communicating (for example through a communication device and/or software, through phone, through video call, face to face as may be determined by analyzing audio captured using an audio sensor such as audio sensor 250, etc.), and so forth.

In some examples, Step 710 may be initiated in response to an action of medical care provider 130. Some examples of such action may include an attempt to access medical records of a patient (such as patient 140), a request for permission to access medical records of a patient (such as patient 140), and so forth. In some examples, process 700 and/or Step 710 may be initiated automatically in response to a detection of electronic device 120 in proximity to electronic device 110 (for example, in the same room, building, in reception range of a radio signal emitted by electronic device 110, in a distance shorter than a selected threshold, and so forth).

In some examples, obtaining information identifying an entity (Step 710) may comprise receiving a signal transmitted by an external device, and in some cases the identifying information and/or the additional information described above may be encoded in the signal. In some examples, electronic device 110 may transmit the signal to electronic device 120. For example, the signal may be transmitted directly from electronic device 110 to electronic device 120 through wireless communication, may be transmitted through a short range wireless communication network, may be transmitted through an intermediate device (such as server 300, cloud platform 400, etc.), may be transmitted through communication network 190, and so forth. Furthermore, electronic device 110 may have access to information identifying medical care providers associated with electronic device 110 (such as medical care provider 130), and the signal may include the identifying information.

In some examples, obtaining information identifying an entity (Step 710) may comprise obtaining image data captured from an environment of the patient and/or the medical care professional (for example obtaining one or more images captured using a mobile image sensor, such as image sensor 260, image sensor included in electronic device 110, image sensor included in electronic device 120, etc.), and analyzing the image data to obtain the identifying information. For example, the image data may be analyzed using face detection algorithms, face recognition algorithms, machine learning algorithm (such as an artificial neural network, a convolutional neural network, a random forest, a support vector machine, etc.) trained to recognize people from images using training examples, and so forth. In another example, the image data may be analyzed to detect and/or recognize a visual identifier (such as a barcode, a QR code, a name tag, and so forth), and the identifying information may be determined from the visual identifier. In some examples, image data captured by electronic device 110 and/or electronic device 120 may be analyzed to detect and/or recognize patient 140 and/or medical care professional 130 from the image data.

In some examples, obtaining information identifying an entity (Step 710) may comprise obtaining audio data captured from an environment of the patient and/or the medical care professional (for example obtaining audio captured using an audio sensor, such as audio sensor 250, audio sensor included in electronic device 110, audio sensor included in electronic device 120, etc.), and analyzing the audio data to obtain the identifying information. For example, the audio data may be analyzed using voice recognition algorithms, machine learning algorithm (such as an artificial neural network, a convolutional neural network, a random forest, a support vector machine, etc.) trained to recognize people from audio using training examples, and so forth. In some examples, audio data captured by electronic device 110 and/or electronic device 120 may be analyzed to detect and/or recognize patient 140 and/or medical care professional 130 from the audio data.

In some examples, obtaining information identifying an entity (Step 710) may comprise obtaining and/or receiving identifying information from an identification device, such as an integrated circuit card (a.k.a. a smart card), a magnetic stripe card, an RFID identifier, an identification card, and so forth. For example, an identification device of patient 140 may provide identifying information of the patient to electronic device 110, to electronic device 120, and so forth. In another example, an identification device of medical care professional 130 may provide identifying information of the medical care professional to electronic device 110, electronic device 120, and so forth.

In some examples, obtaining information identifying an entity (Step 710) may comprise obtaining information identifying the entity from a user (such as patient 140, medical care professional 130, etc.) using an input device, such as a keyboard, a touch screen, a voice input, and so forth. For example, such identifying information may comprise an identification number, a name, an address, a year of birth, a user-name, a password, and so forth.

In some examples, obtaining information identifying an entity (Step 710) may comprise obtaining information identifying the entity from a scheduling system. For example, a schedule for medical care professional 130 may include a meeting with patient 140, and the identity of the patient meeting medical care professional 130 may be determined according to the current time and the scheduled meeting. In another example, medical care professional 130 may be provided with a list of patients scheduled to visit and/or waiting in a waiting area (for example using electronic device 110), medical care professional 130 may select a patient to meet of the list, and the identifying information of the selected patient may be obtained according to the selection.

In some embodiments, medical care provider (such as medical care provider 130) may transmit (for example using electronic device 110) to a server (such as server 300, cloud platform 400, etc.) a request to access medical records of a patient. The request may comprise information identifying the patient (for example the information obtained by Step 710) and/or information identifying the medical care provider. The server may use the received information that identifies the patient to select a mobile device associated with the patient. For example, by accessing a database using the information that identifies the patient to retrieve information, and using the retrieved information to select the mobile device. In another example, a list of one or more mobile devices associated with the patient may be accessed to select the mobile device. In yet another example, a relative of the patient may be selected (for example using a database as described above), and a mobile device associated with the relative of the patient may be selected. In another example, the server may determine that the patient has a legal guardian (for example, using a database, using the age of the patient, using a record of the patient, etc.), and as a result the system may select a mobile device of the legal guardian of the patient. Finally, an indication of the request of the medical care provider to access medical records of the patient may be transmitted to the selected mobile device (for example using communication network 190, from the server, from electrical device 110, and so forth). The indication transmitted to the selected mobile device may comprise information identifying the medical care provider, information identifying the patient, details of the request of the medical care provider, and so forth. In some examples, the transmitted indication may be configured to cause the selected mobile device to provide a request to a user to grant permission to access at least part of the medical records of the patient.

In some embodiments, providing request to a user (Step 720) may comprise providing to the user a request to grant permission to access at least part of the medical records of one or more patients. For example, the user may be a patient, and the medical records may comprise medical records of the user. In another example, the medical records may comprise medical records of one or more family relatives of the user. In yet another example, the user may be a legal guardian of one or more persons, and the medical records may comprise medical records of the one or more persons.

In some examples, providing request to a user (Step 720) may comprise providing the request to the user through a mobile device associated with the user, through a text message, through a voice message, through an email, through a web page, through social network, through a user interface, through a screen, through a sound speaker, through a virtual reality system, through an augmented reality system, and so forth. In some examples, the request provided by Step 720 may comprise a visual request, for example through a visual user interface, such as visual user interface 800 described below. In some examples, the request provided by Step 720 may comprise a textual request, for example by providing textual description of the requested permission to the user. In some examples, the request provided by Step 720 may comprise an audible request, for example by providing an audible description of the requested permission to the user.

In some examples, the request provided by Step 720 may specify one or more of the entities identified by Step 710, identify one or more entities to receive the permission (such as medical care provider 130, a relative of the patient, a legal guardian of the patient, a medical care provider, a medical care institute, an insurer, and so forth), identify patients associated with the medical records (such as patient 140, the user receiving the request, relatives of the user, patients under the custody of the user), identify the user supposed to receive the request, and so forth.

In some examples, the request provided by Step 720 may specify a time period, and the provided request may comprise a request to grant permission to access medical records corresponding to the specified time period. For example, the requested permission may be for medical records of the last specified number of days, months, and/or years. In another example, the requested permission may be for medical records of a given time period that ended in the past. In yet another example, the requested permission may be for medical records of a given time period that ends in the future. In another example, the requested permission may be for medical records that do not correspond to the specified time period, therefore excluding the medical records corresponding to the specified time period from the granted permission. In some examples, the requested permission may be for medical records created in the specified time period, modified in the specified time period, accessed in the specified time period, and so forth.

In some examples, the request provided by Step 720 may specify a time period, and the requested permission may be valid for the specified time period. For example, the requested permission may be valid for a time period starting at the present and ending at a specified time in the future. In another example, the requested permission may be valid from a specified time in the future. In yet another example, the requested permission may be valid from a first specified time in the future and ending in a second specified time in the future, where the second specified time is later than the first specified time.

In some examples, the request provided by Step 720 may specify a type of access for the permission. For example, the requested permission may comprise permission to see the medical record, permission to modify the medical record, permission to add to the medical records, permission to create new medical records, permission to delete the medical records, permission to seal the medical records, and so forth.

In some examples, the request provided by Step 720 may specify a portion of the medical records associated with the patient, and the requested permission may be granted for the specified portion of the medical records. For example, the portion may be specified as a time period as described above. In another example, the portion may comprise medical records corresponding to a specified medical condition, may comprise medical records that do not correspond to a specified medical condition (excluding medical records that corresponds to the specified medical condition from the permission), and so forth. In yet another example, the portion may comprise medical records corresponding to a specified medical field, may comprise medical records that do not correspond to a specified medical field (excluding medical records that corresponds to the specified medical field from the permission), and so forth. In another example, some medical records may correspond to an entity (such as an entity that created the medical records, an entity that modified the medical records, an entity that viewed the medical records, an entity that has permission regarding the medical records, etc.), and the portion may comprise medical records corresponding to a specified entity, medical records not associated with a specified entity (excluding medical records that corresponds to the specified entity from the permission), and so forth. Some examples of such entities may include medical care provider, a medical care institute, an insurer, and so forth.

In some examples, the request provided by Step 720 may specify one or more third parties that the entity that receives the permission may transfer and/or extend the permission to. Some examples of such third parties may include relatives of the patient, legal guardians of the patient, medical care providers, medical care institutes, insurers, and so forth.

In some embodiments, process 700 may identify that a request for a permission was previously denied by a user, and forgo providing an additional request to a user (for example, by forgoing Step 720 and/or other steps of process 700), for example based on time passed since the previous request, based on a similarity between the previous request and the additional request, and so forth. For example, process 700 may forgo providing the additional request when the time elapsed since the previous request is shorter than a selected threshold. In another example, process 700 may forgo providing the additional request when the previous request and the additional request specify the same entity (such as the same medical care provider to receive the permission, the same medical care institute to receive the permission, the same insurer to receive the permission, the medical records of the same patient, and so forth). In yet another example, process 700 may forgo providing the additional request when all the details of the two requests are identical, when a selected portion of the details of the two requests are identical, when a specific detail is identical in the two requests, when all the details of the two requests are similar, when a selected portion of the details of the two requests are similar, when a specific detail is similar in the two requests, and so forth. For example, details may be considered similar when a selected similarity function gives a similarity value higher than a selected similarity value for the details, when a selected distance function gives a distance lower than a selected value for the details, and so forth.

In some embodiments, receiving response from the user (Step 730) may comprise receiving a response from the user to the request provided by Step 720. In some examples, receiving response from the user (Step 730) may comprise receiving the response from the user through a mobile device associated with the patient (such as electronic device 120, the mobile device used to provide the request to the user by Step 720, etc.), through a text message, through a voice message, through a voice command, through an email, through a web page, through social network, through a user interface (such as visual user interface 800 described below), through a microphone, through a keyboard, through a touch screen, and so forth. In some examples, the response received by Step 730 may comprise an audible response, a hand gesture, a key press, a selection in a user interface, a textual response, and so forth.

In some examples, the response received by Step 730 may comprise an identifier of the request provided by Step 720, one or more of the details provided by Step 720, one or more modifications to the details of the request provided by Step 720, a signature of the user providing the response, a digital signature of the user providing the response, a digital signature confirming the integrity of the content of the response, an identifier of the user providing the response, and so forth. In some examples, the response received by Step 730 may comprise an approval to grant permission, a refusal to grant permission, limitations on the permission, details of the permissions, and so forth.

In some examples, the response received by Step 730 may identify one or more entities, indicating the desire of the user to grant permission to the identified one or more entities. Some examples of such entities may include a patient, a relative of the patient, a legal guardian of the patient, a medical care provider, a medical care institute, an insurer, and so forth.

In some examples, the response received by Step 730 may include a specified time period, and indicates the desire of the user to grant permission to access medical records corresponding to the specified time period. For example, the user may desire to grant permission to access medical records of the last specified number of days, months, and/or years. In another example, the user may desire to grant permission to access medical records of a given time period that ended in the past. In yet another example, the user may desire to grant permission to access medical records of a given time period that ends in the future. In another example, the user may desire to grant permission to access medical records that do not correspond to the specified time period, therefore excluding the medical records corresponding to the specified time period from the granted permission. In some examples, the user may desire to grant permission to access medical records created in the specified time period, modified in the specified time period, accessed in the specified time period, and so forth.

In some examples, the response received by Step 730 may specify a time period, and indicates the desire of the user that the grant permission will be valid for the specified time period. For example, the user may desire that the granted permission will be valid for a time period starting at the present and ending at a specified time in the future. In another example, the user may desire that the granted permission will be valid from a specified time in the future. In yet another example, the user may desire that the granted permission will be valid from a first specified time in the future and will expire in a second specified time in the future, where the second specified time is later than the first specified time.

In some examples, the response received by Step 730 may specify a type of access, indicating the desire of the user that the granted permission will be for the specified type of access. For example, the user may desire to grant permission to see the medical record, to modify the medical record, to add to the medical records, to create new medical records, to delete the medical records, to seal the medical records, and so forth.

In some examples, the response received by Step 730 may specify a portion of the records associated with the patient, indicating the desire of the user to grant permission to access the specified portion of the medical records. For example, the portion may be specified as a time period as described above. In another example, the portion may comprise medical records corresponding to a specified medical condition, may comprise medical records that do not correspond to a specified medical condition, and so forth. In yet another example, the portion may comprise medical records corresponding to a specified medical field, may comprise medical records that do not correspond to a specified medical field, and so forth. In another example, some medical records may correspond to an entity (such as an entity that created the medical records, an entity that modified the medical records, an entity that viewed the medical records, an entity that has permission regarding the medical records, etc.), and the portion may comprise medical records corresponding to a specified entity, medical records not associated with a specified entity, and so forth. Some examples of such entities may include a medical care provider, a medical care institute, an insurer, and so forth.

In some examples, the response received by Step 730 may specify one or more third parties, indicating a desire of the user to allow the entity that receives the permission to transfer and/or extend the permission to the one or more third parties. Some examples of such third parties may include relatives of the patient, legal guardians of the patient, medical care providers, medical care institutes, insurers, and so forth.

In some embodiments, process 700 may forgo one or more further steps (such as one or more of Step 740, Step 750 and Step 760) in response to a response received by Step 730 indicating a refusal of the user to grant permission. Additionally or alternatively, process 700 may notify other entities associated with the requested permission (such as the patient, medical care provider 130, entities that requested the permission, entities that were denied the permission, and so forth) about the refusal of the user.

In some embodiments, verifying the user identity (Step 740) may comprise verifying the identity of the user, for example by requesting the user to enter a password, by analyzing the behavior of the user and comparing it a known model of the behavior of the user, by accessing a profile stored on and/or associated with a mobile device (such as electronic device 120), by verifying the patient's finger print, by verifying the patient's bioinformatics, and so forth. In some examples, an authentication sever may be asked, for example through communication network 190, to authenticate the user identity. In some examples, an authentication service may be used to authenticate the user identity, for example using cryptographic tools.

In some embodiments, Step 740 may further verify that the user has authority to grant the permission. In some examples, the identity of the user may be used to access a database and retrieve information about the user, and the retrieved information may be used to determine if the user has the authority to grant the permission. For example, the retrieved information may comprise limitations on the permissions the user may grant. In another example, the retrieved information may comprise information about the relation of the user to the patient, and the determination whether the user has authority to grant the permission may be based on the relation, for example according to one or more rules. Some examples of such rule may include authorizing the user to grant permissions to the medical records of the user, of family members of the user, of patients that allowed the user to grant permissions to their medical records, of patients under the custody of the user, and so forth.

In some embodiments, process 700 may continue to Step 750 or forgo Step 750 based on the response received by Step 730 and/or the result of the verification and/or authentication performed at Step 740. For example, when the response received by Step 730 comprises an approval to grant permission, process 700 may continue to Step 750, while when the response comprises a refusal to grant permission process 700 may forgo Step 750. In another example, when the verification and authentication performed by Step 740 succeed, process 700 may continue to Step 750, while when the verification or authentication fail, process 700 may forgo Step 750. In yet another example, when the response received by Step 730 comprises an approval to grant permission and the verification and/or authentication performed by Step 740 succeed, process 700 may continue to Step 750, while when the response comprises a refusal to grant permission or the verification or the authentication fail, process 700 may forgo Step 750.

In some embodiments, granting permission (Step 750) may comprise granting permission to an entity (such as a patient, a relative, a medical care provider, a medical care institute, an insurer, etc.). In some examples, the granted permission may comprise permission to create and/or delete and/or read and/or edit at least part of the records (such as medical records 610, scheduling records 620, financial records 630, insurance records 640, permissions 650, etc.) associated with one or more patients. In some examples, Step 750 may comprise registering the permission with a computerized device (such as electronic device 110, electronic device 120, apparatus 200, server 300, computational node 500, cloud platform 400, etc.), in storage devices (such as remote storage, network attached storage, etc.), in a database, in a blockchain, and so forth. In some examples, Step 750 may comprise transmitting information related to the permission to an external device, for example in order to grant the permission, to record the granted permission, to inform an entity and/or a device and/or a process about the granted permission, and so forth. For example, Step 750 may comprise transmitting one or more messages configured to cause an external device to grant the permission. The one or more messages may comprise details of the permission to be granted. For example, the one or more messages may be transmitted through communication network 190 (for example from a mobile device such as electronic device 110 and/or electronic device 120 to an external device such as server 300, cloud platform 400, etc.), through a short range wireless communication network (for example from electronic device 120 to electronic device 110), and so forth.

In some examples, the permission granted by Step 750 may comprise one or more limitations on the permission, for example based on limitations included in the request provided by Step 720 and/or in the response received by Step 730. For example, the limitation may comprise a limitation of the permission to selected entities, such as the one or more entities specified in the request provided by Step 720 and/or in the response received by Step 730. Some examples of such entities may include a patient, a relative of the patient, a legal guardian of the patient, a medical care provider, a medical care institute, an insurer, and so forth. In another example, the limitation may comprise a limitation of the permission to medical records corresponding to a selected time period, to medical records that do not correspond to a selected time period, and so forth. The selected time period may be specified in the request provided by Step 720 and/or in the response received by Step 730. In yet another example, the limitation may comprise a limitation of the permission to selected type of access to the medical records. The selected type of access may be specified in the request provided by Step 720 and/or in the response received by Step 730. Some examples of such types of accesses may include viewing the medical records, modifying the medical record, adding to the medical records, creating new medical records, deleting the medical records, sealing the medical records, and so forth.

In some examples, the permission granted by Step 750 may be limited to a selected portion of the medical records associated with the patient. The selected portion of the medical records may be specified in the request provided by Step 720 and/or in the response received by Step 730. For example, the selected portion may be specified as a time period as described above. In another example, the selected portion may comprise medical records corresponding to a specified medical condition, may comprise medical records that do not correspond to a specified medical condition, and so forth. In yet another example, the selected portion may comprise medical records corresponding to a specified medical field, may comprise medical records that do not correspond to a specified medical field, and so forth. In another example, some medical records may correspond to an entity (such as an entity that created the medical records, an entity that modified the medical records, an entity that viewed the medical records, an entity that has permission regarding the medical records, etc.), and the selected portion may comprise medical records corresponding to a specified entity, medical records not associated with a specified entity, and so forth. Some examples of such entities may include medical care provider, a medical care institute, an insurer, and so forth.

In some examples, the permission granted by Step 750 may be valid for a selected time period. The selected time period may be specified in the request provided by Step 720 and/or in the response received by Step 730. For example, an expiration time for the permission may be specified and/or indicated in the request provided by Step 720 and/or in the response received by Step 730. In some examples, the user may be notified about an approaching expiration of the granted permission, may be notified before the expiration of the permission, may be notified at the time of expiration, may be notified after the expiration of the permission, and so forth. A request indicating a desire of the user to extend the permission may be received, for example through a mobile device associated with the patient, through a text message, through a voice message, through a voice command, through an email, through a web page, through social network, through a user interface (such as visual user interface 800 described below), through a microphone, through a keyboard, through a touch screen, and so forth. In response to the indication of the desire of the user to extend the permission, the permission may be extended beyond the planned expiration time. For example, the user may indicate a desire to extend the permission for additional selected time period, to delay the expiration time by selected time duration, to turn the permission to a permanent permission that has no expiration time, and so forth.

In some examples, the permission granted by Step 750 may allow a recipient of the permission to extend the permission or part of the permission to third parties. For example, the third parties that the recipient of the permission may extend the permission to may be specified in the request provided by Step 720 and/or in the response received by Step 730. Some examples of such third parties may include relatives of the patient, legal guardians of the patient, medical care providers, medical care institutes, insurers, and so forth.

In some embodiments, a type of a medical care provider and/or a medical care institute may be used to automatically select at least one limitation to a permission. For example, the permission may be limited to a portion of the medical records based on the specialization of the medical care provider, for example limiting the permission to records dealing with medical conditions associated with the specialization, limiting the permission to exclude some sensitive medical information not related to the specialization, and so forth. In another example, the duration of the permission may be limited according to the type of medical care institute, for example limiting the permission to a short duration for an emergency room, avoiding time limitations for family health centers, and so forth. In some examples, the selected limitations may be suggested to the user by Step 720, and the user may accept the suggested limitations, modify the suggested limitations, override the suggested limitations, and so forth. In some examples, the selected limitations may be added automatically to the permission granted by Step 750.

In some embodiments, informing users (Step 760) may comprise informing users about one or more intermediate and/or final result of process 700. Some examples of such users may include patients, relatives of patients, legal guardians of patients, medical care providers, medical care institutes, insurers, the user of Step 720, the user of Step 730, and so forth. In some examples, informing users (Step 760) may comprise informing the users that a permission was granted, that a permission was not granted, that an error occurred while performing process 700 and/or while granting the permission (possibly with information about the error), that the verification and/or authentication performed by Step 740 failed, and so forth. In some example, informing users (Step 760) may comprise informing the users through a mobile device associated with the patient, through a text message, through a voice message, through an email, through a web page, through social network, through a user interface, through a screen, through a sound speaker, through a virtual reality system, through an augmented reality system, and so forth. In some examples, the information provided by Step 760 may be provided visually, audibly, textually, and so forth.

In some examples, the information provided by Step 760 may identify one or more entities that received the permission by Step 750. Some examples of such entities may include a patient, a relative, a medical care provider, a medical care institute, an insurer, and so forth. In some examples, the information provided by Step 760 may specify a time period associated with the permission granted by Step 750. In some examples, the information provided by Step 760 may specify a type of access associated with the permission granted by Step 750. Some examples of such type of access may include read, create, delete and edit. In some examples, the information provided by Step 760 may specify at least one portion of the records associated with the patient related to the permission granted by Step 750. In some examples, the information provided by Step 760 may specify one or more third parties that the entity that received the permission granted by Step 750 may transfer and/or extend the permission to.

In some embodiments, Step 760 may comprise providing a list of granted permissions to the user. For example, a patient may be provided with a list of permissions to access the patient's medical records, a user may be provided with a list of permissions to access medical records of entities related to the user (such as relatives of the users, people under the custody of the user, patients of the user, etc.), and so forth. Further, an indication of at least one desired modification to at least one permission (for example, of the list of granted permissions that was provided to the user) may be received from the user. Some examples of the at least one desired modification may include a desire to cancel a permission, a desire to change a limitation of the permission (such as the limitations described above), a desire to add a new limitation to the permission (such as the limitations described above), a desire to cancel a limitation to the permission (such as the limitations described above), and so forth. In response to the indication of at least one desired modification received from the user, process 700 may modify the permission according to the at least one desired modification.

In some embodiments, Step 760 may comprise providing a list of permissions requests denied by a user (for example using Step 730) to users. For example, a patient may be provided with a list of denied permissions requests associated with the patient's medical records, a user may be provided with a list of denied permissions requests associated with entities related to the user (such as relatives of the users, people under the custody of the user, patients of the user, etc.), and so forth. Further, an indication of a desire of the user to grant previously denied permissions (for example, of the list of denied permission requests that was provided to the user) may be received from the user. In response to the indication of a desire of the user to grant previously denied permissions, process 700 may grant the previously denied permission, for example using Step 750.

In some examples, permissions grated by Step 750 may be registered in a log. Moreover, additional information associated with the granted permissions may also be registered in the log, such as details of the granted permission, time of granting, information about communication with the user by Step 720 and/or Step 730, and so forth. Additionally or alternatively, accesses to medical records may be registered in the log. Information associated with the accesses to the medical records may also be registered, such as access type, access time, accessing entity, accessed portion of the medical records, and so forth. In some examples, the log may be analyzed to generate a report, and the report may be provided to a user, to a patient, to a medical care provider, to a medical care institute, to an insurer. For example, a report about granted permissions associated with medical records of a patient and/or about accesses to medical records of the patient may be provided to the patient, a family relative of the patient, a legal guardian of the patient, a medical care provider of the patient, an insurer of the patient, and so forth.

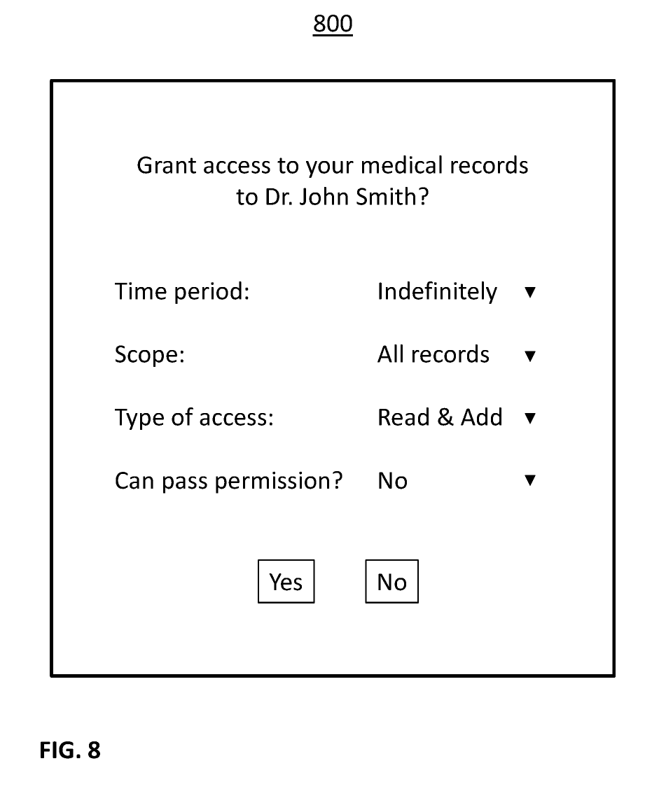

FIG. 8 illustrates an example of visual user interface 800. For example, visual user interface 800 may be generated by Step 720, and inputs entered by a user in visual user interface 800 may be received as a response by Step 730. In this example, visual user interface 800 may comprise a request to approve granting permission. In this example, visual user interface 800 may also comprise an identification of the entity that will receive the permission, in this case identifying the entity as Dr. John Smith. In other examples, the entity to receive the permission may also be identified by a Physician's License Number, by an image of the person, and so forth. In this example, visual user interface 800 may also comprise parameters of the permission (such as time period, scope, type of access, permission to pass and/or extend the permission, etc.), and a user of visual user interface 800 may change the parameters of the permissions. In this example, visual user interface 800 may also comprise ‘Yes’ and ‘No’ buttons, and a user of visual user interface 800 may press the ‘Yes’ button to approve granting the permission, or press ‘No’ to refuse to grant permission.

It will also be understood that the system according to the invention may be a suitably programmed computer, the computer including at least a processing unit and a memory unit. For example, the computer program can be loaded onto the memory unit and can be executed by the processing unit. Likewise, the invention contemplates a computer program being readable by a computer for executing the method of the invention. The invention further contemplates a machine-readable memory tangibly embodying a program of instructions executable by the machine for executing the method of the invention.

What is claimed is:

1. A mobile device for enabling patients to control access to their medical records, the mobile device comprising:
at least one communication unit; and
at least one processing unit configured to:
use the at least one communication device to receive information identifying one or more medical care providers;
provide to a patient a request to grant permission to the one or more medical care providers to access at least part of a plurality of medical records of the patient;
receive a response to the request from the patient; and
based on the response, use the at least one communication device to transmit a message configured to cause the permission to access the at least part of the plurality of medical records of the patient to be granted to at least part of the one or more medical care providers.

2. A method for enabling patients to control access to their medical records, the method comprising:
receiving at a mobile device associated with a patient, information identifying one or more medical care providers;
using the mobile device associated with the patient to provide to a user a request to grant permission to the one or more medical care providers to access at least part of a plurality of medical records of the patient;
using the mobile device associated with the patient to receive a response to the request from the user; and
based on the response, granting the permission to at least part of the one or more medical care providers to access the at least part of the plurality of medical records of the patient.

3. The method of claim 2, further comprising:
receiving at a server a request from at least one of the one or more medical care providers to access the at least part of a plurality of medical records of the patient, the request received at the server comprises information identifying the patient;
using the information identifying the patient to access a database to select the mobile device associated with the patient; and
transmitting from the server to the mobile device associated with the patient the information identifying the one or more medical care providers.

4. The method of claim 3, wherein the mobile device associated with the patient is a mobile device of a legal guardian of the patient.

5. The method of claim 2, further comprising:
using the mobile device to capture image data from an environment of the user; and
analyzing the image data to obtain the information identifying the one or more medical care providers.

6. The method of claim 2, further comprising:
using a short range wireless communication network to transmit from a device associated with the one or more medical care providers to the mobile device associated with the patient the information identifying the one or more medical care providers; and
based on the response, using the short range wireless communication network to transmit from the mobile device associated with the patient to the device associated with the one or more medical care providers a signal configured to cause the granting of the permission.

7. The method of claim 2, further comprising:
determining that time elapsed since a previous request was provided to the user is shorter than a selected threshold; and
based on said determination, forgoing providing the request to the user.

8. The method of claim 2, further comprising:
determining that a similarity of the received identifying information to at least one medical care provider corresponding to a previous request provided to the user is higher than a selected threshold; and
based on said determination, forgoing providing the request to the user.

9. The method of claim 2, wherein at least one of the request and the response comprises an indication of a time period, and wherein the granted permission is limited to medical records of the indicated time period.

10. The method of claim 2, wherein at least one of the request and the response comprises an indication of an expiration time, and wherein the granted permission is configured to expire according to the indicated expiration time.

11. The method of claim 10, further comprising:
notifying the user about an approaching expiration of the granted permission;
receiving from the user a request to extend the granted permission; and
based on the received request to extend the granted permission, extending the granted permission beyond the indicated expiration time.

12. The method of claim 2, wherein at least one of the request and the response comprises an indication of a type of access, and wherein the granted permission comprises a limitation based on the type of access.

13. The method of claim 2, further comprising:
using a type of the one or more medical care providers to select a limitation to the permission; and
limiting the granted permission according to the selected limitation.

14. The method of claim 2, wherein the granted permission further comprises a permission to the at least part of the one or more medical care providers to provide access to the at least part of the plurality of medical records of the patient to at least one of an additional medical care provider, a relative of the patient and an insurer.

15. The method of claim 2, wherein granting the permission comprises registering the granted permission in a blockchain.

16. The method of claim 2, further comprising:
using an authentication server to attempt to verify an identity of the user; and
in response to a failure to verify the identity of the user, forgoing granting the permission.

17. The method of claim 2, further comprising:
registering the granted permission in a log;
registering accesses to the plurality of medical records in the log;
analyzing the log to generate a report; and
providing the report to the patient.

18. The method of claim 2, further comprising:
providing a list of granted permissions to the user;
receiving from the user at least one modification to at least one permission of the list of granted permissions; and
in response to the received at least one modification, modifying the at least one permission of the list of granted permissions.

19. The method of claim 2, further comprising:
providing to the user a list of permissions requests denied by the user;
receiving from the user an indication of a desire of the user to grant at least one previously denied permission of the list of permissions requests denied by the user; and
in response to the received indication, granting the at least one previously denied permission.

20. A non-transitory computer readable medium storing a software program comprising data and computer implementable instructions for carrying out a method for enabling patients to control access to their medical records, the method comprising:
receiving at a mobile device associated with a patient, information identifying one or more medical care providers;
using the mobile device associated with the patient to provide to a user a request to grant permission to the one or more medical care providers to access at least part of a plurality of medical records of the patient;
using the mobile device associated with the patient to receive a response to the request from the user; and
based on the response, granting the permission to at least part of the one or more medical care providers to access the at least part of the plurality of medical records of the patient.
